(19) United States
(12) Patent Application Publication
(10) Pub. No.: US 2025/0224386 A1
(43) Pub. Date: Jul. 10, 2025

(54) DIFFUSION DISCRIMINATING GAS SENSORS

(71) Applicant: KATHOLIEKE UNIVERSITEIT LEUVEN, Leuven (BE)

(72) Inventors: Rob AMELOOT, Leuven (BE); Aleksander MATAVŽ, Ravne na Koroškem (SI)

(21) Appl. No.: 18/852,781

(22) PCT Filed: Apr. 3, 2023
(86) PCT No.: PCT/EP2023/058622

§ 371 Date: Sep. 30, 2024

(30) Foreign Application Priority Data
Apr. 1, 2022 (EP) 22020147.9

(51) Int. Cl. G01N 33/00; B82Y 15/00; G01N 5/02; G01N 21/41; G01N 27/22

(52) U.S. Cl. CPC G01N 33/0047; G01N 5/025; G01N 21/41; G01N 27/221; G01N 27/227; B82Y 15/00; G01N 2021/4126; G01N 2027/222; G01N 27/223

(57) ABSTRACT

The invention relates to a gas sensor for detecting one or a plurality of volatile compounds in a gas, the sensor comprising: —a signal transducer comprising a layer comprising nanopores wherein the gas has access to the nanopores in said layer, —a heating or cooling element, with adjustable temperature settings, for heating or cooling said layer comprising said nanopores, —means for access of said gas to said layer comprising said nanopores, —an electronic circuit, monitoring a time-dependent and a temperature-dependent signal generated by the signal transducer upon adsorption or release of a compound from the nanopores in said layer.

### FIELD OF THE INVENTION

The present invention relates to gas sensors and particularly to sensors capable of sensitive and selective detection of gases.

### BACKGROUND

Gas sensing is a rapidly developing technology field due to its vast application potential in the monitoring of air polluting levels (indoors, outdoors, or in industrial applications), food freshness, healthcare diagnostics (via breath analysis), etc. At present, gas sensing technology is dominated by metal oxide semiconductor (MOS) sensors, which often lack selectivity and cannot efficiently distinguish between gaseous components, in particular between volatile organic compounds (VOCs). Hence, a novel miniaturized sensor technology capable of distinguishing a VOC of interest from a complex background is needed to reach the full potential in the above-mentioned applications.

An emerging class of gas sensors are adsorption-based sensors, which employ a microporous sensing layer made of metal-organic frameworks (MOFs) or zeolites. Herein, MOFs will be addressed in more detail, but the concepts can be equally applied to other microporous or nanoporous materials. MOFs are a class of crystalline solids that consist of inorganic nodes connected by organic linkers [Kitagawa et al. (2004) Angew. Chem. Int. Ed. 43, 2334-2375]. Uniform nanopores (typically 0.5-2 nm) and walls of only a single molecule thick form through self-assembly of the MOF crystal lattice and give rise to record internal surface areas (up to >6000 m²g⁻¹) [Farha et al. (2012) J. Am. Chem. Soc. 134, 15016-15021]. Because of their chemical and structural features, MOFs can capture analytes, such as VOCs, even at trace concentrations, with partition coefficients orders of magnitude higher than established materials (Leidinger et al. (2016) Sensors and Actuators B: Chemical 236, 988-996). So far, MOF-based sensing has been exclusively based on the equilibrium sensor response and, therefore, relied only on thermodynamic adsorption parameters to achieve selectivity (i.e., differences between equilibrium uptake isotherms).

US20180195990 discloses a gas sensor comprising a gas-sensing material including a metal-organic framework with fcu topology (RE-fcu-MOF), wherein the ligand of the RE-fcu-MOF is one or more of fumaric acid and 1,4-napthalene dicarboxylic acid. The sensing signal transduction was based on measuring the capacitance of a sensor in interdigitated electrode configuration. Sensors showed higher sensitivity to H₂S and NH₃ vapours when compared to NO₂, CH₄, H₂ and C₇H₈.

Campbell et al. (2015) J. Am. Chem. Soc. 137, 13780-13783, demonstrated a chemiresistive sensor array from conductive 2D metal-organic frameworks. The array consisted of three separate devices, each employing a different MOF as a sensing layer. The combined response of the sensor array was treated using principal component analysis, which yielded classification of the data by functional group class (alkanes, alcohols, ketones, amines, aromatics and aliphatics).

Alternative to sensor arrays, a multivariable approach was used to improve the selectivity of a single sensor element [Potyrailo (2016) Chem. Rev. 116, 11877-11923]. In contrast to traditional single-output sensors, a single multivariable sensor generates two or more (at least partially) independent outputs. The non-correlated information in these outputs improves the selectivity compared to single-output sensors and can enable quantification of the individual VOCs in a mixture by using standard methods used in chemometrics (e.g., principal component analysis). Moreover, since the multivariable sensor makes use of a single sensing material, the drift of the outputs (if any) is correlated and can be more easily compensated for. The multivariable sensors based on MOFs have been up to now only reported for optical transduction, for example, measuring the wavelength-dependent adsorption of MOF [Potyrailo, cited above].

### SUMMARY OF THE INVENTION

Described in this invention are multivariable gas sensors that rely on the discrimination of analytes based on their diffusion kinetics. The diffusion constant in microporous materials can vary over orders of magnitude for different analytes since their diffusion is controlled by steric hindrance and specific host-analyte interactions. Therefore, even molecules with similar uptake properties can often be discriminated based on diffusion kinetics. For example, methanol and ethanol diffuse dramatically faster in ZIF-8 (a microporous MOF material) compared to 1-butanol, leading to an ideal kinetic selectivity of up to 10⁶, while the thermodynamic selectivity is modest at comparable concentrations. Similarly, while the uptakes of butanol and benzene are similar, the diffusion of benzene is >10⁴ times slower. In present invention “kinetic selectivity” is defined as the ratio of the diffusivities of the adsorbates, and “thermodynamic selectivity” as the ratio of their equilibrium uptake.

MOF refers to a class of crystalline solids that consist of inorganic nodes connected by organic linkers.

The methods of the present invention can be used for example in the monitoring of air polluting levels (indoors, outdoors, or in industrial applications), food freshness, healthcare diagnostics (via breath analysis).

Throughout the specification nanopores or nanoporous are used as synonyms for micropores or microporous.

The invention is further summarised in the following statements:

1. A gas sensor for detecting one or a plurality of volatile compounds in a gas, the sensor comprising:
a signal transducer comprising a layer comprising nanopores wherein the gas has access to the nanopores in said layer,
a heating or cooling element, with adjustable temperature settings, for heating or cooling said layer comprising said nanopores,
means for access of said gas to said layer comprising said nanopores,
an electronic circuit, monitoring a time-dependent and a temperature-dependent signal generated by the signal transducer upon adsorption or release of a compound from the nanopores in said layer.

“Volatile compound” refers to a compound which, at room temperature, occur partially or completely, in gas phase. Examples thereof include water, alkanes, ketones, alcohols, aldehydes, and aromatic compounds.

Herein water is typically a background component that hampers measurements of other volatile components.

2. The gas sensor according to statement 1, wherein the layer comprising nanopores has a thickness of less than 50 μm, or of less than 10 μm, or less than 5 μm, for example between 50 to 300 nm.

3. The gas sensor according to statement 1 or 2, wherein the nanopores in the layer comprising nanopores have an average diameter of below 10 nm, of below 10 nm, or of below 2 nm.

4. The gas sensor according to any one of statements 1 to 3, wherein the layer comprising nanopores is a zeolite or a porous carbon.

5. The gas sensor according to any one of statements 1 to 3, wherein the layer comprising nanopores is a MOF (Metal-Organic Framework).

6. The gas sensor according to any one of statements 1 to 5, wherein the heating element and layer comprising nanopores are separated by a heat conductive material.

7. The gas sensor according to statement 6, wherein the heat conductive material has a heat conductivity of at least 0.3 W/mK.

8. The gas sensor according to statement 6 or 7, wherein the heat conductive material is a non-electrically conductive material such as silicon nitride, silicon oxide, silicon carbide or a ceramic.

9. The gas sensor according to any one of statements 1 to 8, wherein the heating element is ohmic heater, such as a micro hotplate.

10. The gas sensor according to any one of statements 1 to 9, wherein the signal transducer is an electronic, capacitive, optical or gravimetric signal transducer.

11. The gas sensor according to statement 10, wherein the capacitive signal transducer comprises a bottom heat conductive layer, and a top gas permeable conductive layer, and the layer comprising nanopores is positioned between said bottom layer and said top layer.

12. The gas sensor according to statement 10, wherein the optical signal transducer a bottom reflective or semi-reflective layer, a top reflective or semi-reflective gas permeable layer and the layer comprising nanopores is positioned between said bottom and said top layer.

13. The gas sensor according to statement 10, comprising a gravimetric signal transducer wherein the layer comprising nanopores is positioned, and in contact with, on one or more mechanical resonators of which the resonant frequency or amplitude can be monitored.

14. The gas sensor according to statement 13, wherein the gravimetric transducer operates in a static or resonant mode.

15. The gas sensor according to statement 13 or 14, wherein the gravimetric transducer is a cantilever or a coupled resonator.

16. The gas sensor according to any one of statements 1 or 15, wherein the layer comprising the nanopores is in direct contact with the transducer.

17. The gas sensor according to any one of statements 1 or 15, wherein the layer comprising the nanopores and the transducer are spatially separated.

18. The gas sensor according to statement 17, wherein the transducer is a metal oxide semiconductor sensor.

19. The sensor according to any one of statements 1 to 18, wherein the gas sensor comprises a plurality of layers comprising nanopores, wherein the material of the layers have different affinities for a volatile compound and/or different diffusion properties for a volatile compounds, and wherein each of layers is part of an individual signal transducer.

20. The sensor according to statement 19, wherein each of the plurality of layers comprising nanopores can be subjected to a separate temperature regime.

21. The sensor according to statement 19 or 20, wherein each of the plurality of layers comprising nanopores differs in thickness.

22. A method for determining the presence and/or quantity of a plurality of volatile compounds in a gas comprising the steps of:
a) Introducing a gas into a sensor in accordance to any one of statement 1 to 20 whereby compounds in the gas can adsorb in the nanopores of the layer comprising nanopores,
b) decreasing or increasing the temperature of the layer comprising nanopores, thereby releasing adsorbed compounds from the nanopores upon heating, or adsorbing compounds in the nanopores upon cooling,
c) measuring from the signal transducer the temperature-dependent and time dependent release and/or adsorption of compounds from or to the nanopores,
d) determining based on the measurements of the transducer the presence and/or concentration of at least two volatile compounds in the gas.

23. The method according to statement 22, wherein is step b) the temperature of the layer comprising nanopores is increased, thereby releasing adsorbed compounds from the nanopores.

24. The method according to statement 22 or 23, determining in step d) the presence and concentration of water in the gas.

25. The method according to any one of Statements 22 to 24, wherein the temperature of the layer comprising nanopores is perturbated in a periodic manner.

26. The method according to any one of Statements 22 to 25, wherein the adsorption or release of compounds is monitored on multiple layers comprising 30 nanopores.

27. The method according to any one of statements 22 to 26, wherein the gas introduced in step a) contains up to 50% (v/v) up to 75% (v/v) or up to 100% (v/v) water vapor.

28. The method according to any one of statements 22 to 27, wherein the gas introduced in step a) is outside ambient air or air within a building.

29. The method according to any one of statements 22 to 28, wherein the gas introduced in step a) is an exhaled animal or human breath.

30. The method according to any one of statements 22 to 29, wherein the method determines the presence and/or concentration of one or more 1-propanol, 1-butanol, acetone pentane and hexane in a gas.

31. The method according to any one of statements 22 to 30, wherein the method determines the presence and/or concentration of one or more 1-propanol, 1-butanol, acetone pentane and hexane in a gas comprising water vapor.

### DESCRIPTION OF THE INVENTION

### Figure Legends

FIG. 1: (a) A simplified structure of a diffusion discriminating gas sensor. (b) Time-domain detection concept. The sensor with a microporous active layer is rapidly heated from T₁ to T₂ at time t₀. As a result, analyte molecules diffuse out of the microporous layer, which is detected as a signal S by the sensor transducer. The signal follows an exponential decay function from S₂ to S₁ upon analyte desorption. (c) Frequency-domain detection concept. The periodic temperature perturbation with an amplitude T and a frequency v modulates the diffusion in the microporous layer. The sensor signal follows the temperature perturbation with an amplitude S and a phase ϕ. Sweeping the frequency of temperature perturbation produces a step-wise amplitude decrease and a peak in phase lag of sensor signal, that is characteristic for an analyte diffusion. All examples in the figure are for the ideal case of single analyte diffusion.

FIG. 2: Possible configurations of sensor transducer: (a) a top-view projection and (b) a cross-section of a sensing element in the metal-insulator-metal (MIM) configuration, (c) a top-view projection and (d) a cross-section of a sensing element in the interdigitated electrode (IDE) configuration. 1: heater, 2: insulation/substrate, 3: microporous sensing layer, 4: electrode.

FIG. 3: Possible configurations of the thermal transducer. 1: heating element, 2: electric shield, 3: insulation

FIG. 4: Calculated desorption profiles for (a) 50 nm thick film and (b) 1000 nm thick film of microporous material. The discrimination power for binary mixtures of analytes with a factor of (c) 10, (d) 100, and (e) 1000 difference in diffusion constants (calculated for a 50 nm thick film of microporous material). A1/A2 is the concentration ratio of both analytes.

FIG. 5: (a-e) A MIM capacitive sensor with 260 nm thick ZIF-8 layer. (a) The dependence of diffusion signal amplitude on a concentration of 1-butanol for a temperature step of 16° C. (b) The dependence of diffusion signal time constant on a concentration of 1-butanol for a temperature step of 16° C. (c) The dependence of diffusion signal amplitude on a temperature step amplitude for ethanol at p/p⁰=0.1% (at 24° C.). (d) The temporal response of a sensor to methanol (p/p⁰=1% at 24° C.), 1-propanol (p/p⁰=0.1% at 24° C.), 1-butanol (p/p⁰=0.1% at 24° C.), and hexane (p/p⁰=0.05% at 24° C.) upon a temperature step of 16° C. (e) The temporal response of a sensor to a mixture of methanol (p/p⁰=8% at 24° C.) and 1-butanol (p/p⁰=0.5% at 24° C.) upon a temperature step of 16° C. (f) Calculated temporal response of a sensor with 50 nm thick ZIF-8 layer to a mixture consisting of 40% of relative humidity (water), 200 ppm of acetone, and 10 ppm of 1-hexane.

FIG. 6: Multivariable VOC classification using diffusion discriminating sensing approach with a MIM capacitive sensor. Single component and binary mixture measurements on (a) 1-propanol:1-butanol system, (b) acetone:hexane system. (c) Single component it and ternary mixture measurements on water:acetone:hexane system. The concentration of single component in ppm is indicated next to the data points. The measurements were performed at 24° C. using an MIM capacitive sensor with 180 nm thick ZIF-8 layer and 12 nm thick gold top electrode.

FIG. 7: Separated adsorption and measurement elements & sampled measurement method.

A. Open-top implementation of the separated element concept. Dark elements (1): heater with nanoporous material coated on top. Lighter elements (2): transduction element. Top: top view. Bottom: cross-sectional side view.

B-C. Semi-enclosed implementations of the separated element concept. Dark elements (1): heater with nanoporous material coated on top. Lighter elements (2): transduction element. Top: top view. Bottom: cross-sectional side view.

D Sampled measurement method. The adsorbent temperature jumps from T₁ to T₀ (T₀<T₁), and molecules will start to diffuse into the adsorbent (since the adsorbed quantity n₀>n₁). Before equilibrium at T₀ is established, the temperature jumps back to T₁ after a well-defined dwell time t₁, and the molecules that could diffuse in are desorbed. The desorbed fraction is quantified downstream using a mass spectrometer. By systematically varying the time t, the amount adsorbed vs. time curve can be constructed and fitted with a suitable diffusion model to extract the diffusivity at T₀.

The invention presented herein devises a method to selectively detect analytes and measure their concentration by gas sensors or sensor arrays that display kinetic selectivity.

The features disclosed in the description, or in the claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Sensing Principle

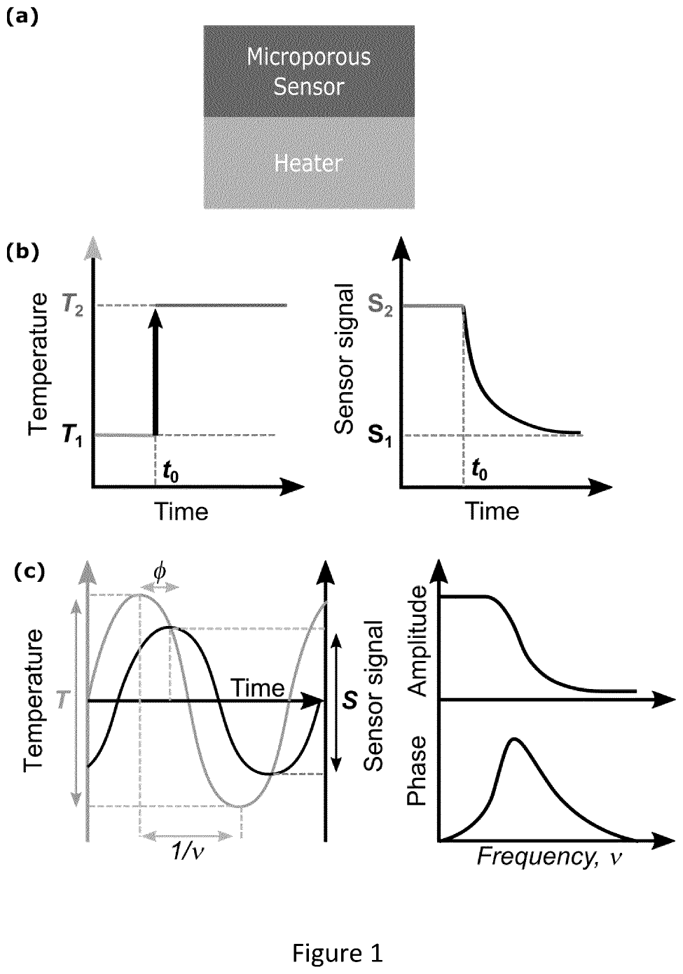

The measurement of diffusion requires out-of-equilibrium conditions for adsorption. The adsorption equilibrium can be modulated by changing the analyte concentration, gas pressure or sensor temperature. The analyte concentration and gas pressure are difficult to modulate in real-world applications, but the temperature modulation is easily achieved by introducing a heating element to the sensor. Therefore, the diffusion discriminating sensors comprise a heating element that provides a rapid temperature perturbation to the sensing element that measures the amount of adsorbed gas (FIG. 1a). The temperature perturbation produces thermodynamic out-of-equilibrium condition for adsorption, which drives the diffusion of analytes until the equilibrium at the given temperature is reached.

Ideally, the microporous layer has a geometrical form that ensures fast diffusion time constants and a single-exponential diffusion event for a single analyte. Possible geometrical forms are thin film and particles with narrow particle size distribution. A particularly suitable form is a thin film. For a simple signal interpretation, the sensor signal is linearly proportional to the number of adsorbed molecules. All examples here assume geometrical form of a thin film and linear relation between signal and amount of adsorbed molecules. In such a case, the diffusion of a single analyte produces an exponential decay function (Equation 1).

$$S ( t ) = \frac{8}{{π}^{2}} {\sum }_{i = 0}^{∞} \frac{1}{{( 2 i + 1 )}^{2}} exp [ - {( 2 i + 1 )}^{2} \frac{{π}^{2}}{4} \frac{D}{{l}^{2}} t ] , \qquad ( Equation 1 )$$

where S is the sensor signal, D is the diffusion constant, l is the film thickness and t is time.

In the first example, the heating element provides a step-like temperature perturbation from temperature T₁ to T₂ (FIG. 1b). The time dependence of the amount of adsorbed molecules is monitored by the signal of a sensing element. In most microporous materials, the increase in temperature results in the desorption of gas molecules. In the case of a single analyte, the sensor signal follows the temperature step with an exponential decay function. The time constant of the exponential decay function is proportional to the diffusion constant of the analyte at temperature T₂. Moreover, the heat of diffusion can be measured by performing the diffusion measurement at different temperatures.

In the second example, the heating element provides a periodic temperature perturbation, for example, in the form of a sine wave (FIG. 1c). The frequency of temperature perturbation is swept across a frequency range and the sensor signal at each frequency is monitored. At a much lower frequency than the analyte diffusion time constant, the sensor signal follows the temperature perturbation without a phase lag. The amplitude of signal relates to the amount of adsorbed analyte. When the temperature perturbation frequency approaches the diffusion time constant, the sensor signal phase lag increases until it reaches a peak and the amplitude decreases in a step-wise fashion. At a frequency much higher than the diffusion time constant, the sensor signal again follows the temperature perturbation without a phase lag, but the sensor amplitude corresponds to the response of the empty sensing layer (i.e., the signal relates to the temperature-dependence of dielectric properties of the sensing layer). The main advantage of using the periodic temperature perturbation is the possibility of using phase-sensitive detection methods, such as detection of diffusion signal using lock-in amplifier. If necessary, the non-linear contributions due to thermal activation of diffusion can be avoided by using sufficiently small temperature perturbation amplitude. Phase-sensitive detection methods can significantly improve the signal-to-noise ratio and improve the detection limit of the sensor.

### Sensor Configurations

A sensor transduction mechanism translates the amount of analyte adsorbed to measurable signal. Possible transduction mechanisms include the ones with electrical, optical or gravimetrical readout. Especially useful are electrical and optical readouts, since they can be easily integrated with a heater element. A possible transduction mechanism for adsorption sensors with electrical readout is the measurement of capacitance (or complex impedance), since most of the microporous materials are dielectrics. Upon the adsorption of analyte molecules in the microporous material, its effective dielectric constant increases proportionally to the amount adsorbed and the dielectric constant of the adsorbed phase. Similarly, the refractive index increases upon adsorption of analytes, which is exploited in sensors with optical readout. Here, the capacitive sensors are described in more detail, although same principles apply also to sensors with other transduction mechanisms.

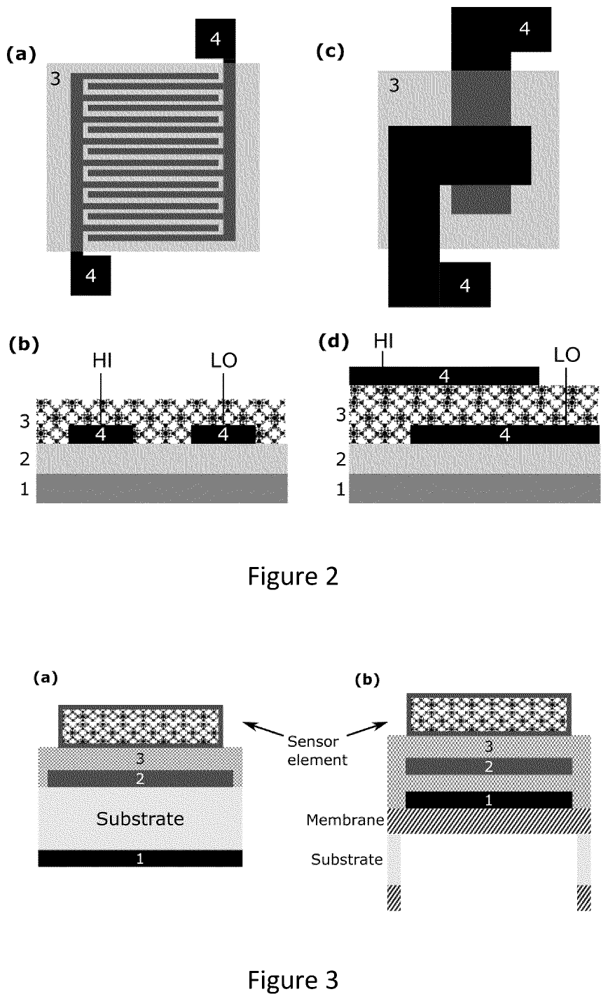

Suitable capacitor architectures are the metal-insulator-metal (MIM) configuration (FIG. 2a,b) and the interdigitated electrode (IDE) configuration (FIG. 2c,d). Both configurations are described in detail below.

The MIM configuration consists of a microporous sensing layer sandwiched between two electrodes. The capacitance of the MIM senor is determined by the area of electrode overlap, the thickness of the sensing layer and dielectric constant of the sensing layer. The bottom electrode is deposited on the substrate and is covered by a microporous sensing layer. The top electrode is deposited on the top of a sensing layer and is gas permeable. In some embodiments, the top electrode is in the form of a thin layer made of noble metal (Au, Pt, or Ag) with thickness between 7-100 nm. In the MIM configuration, the top electrode can, in some cases, introduce an additional resistance for the diffusing gas molecules, which increases the apparent diffusion time constants. The diffusion resistance of the electrode scales with the denser morphology of the electrode (or absence of pinholes in the electrode) and, consequently, with its thickness. In some embodiments, the electrode thickness is used to shift the diffusion time constants of fast-diffusing analytes into the operating range of a sensor by increasing the diffusion path length through the microporous material.

The IDE configuration consists of in-plane electrodes in the form of interdigitated fingers separated by a certain distance. The sensing layer is deposited on top of the electrodes, or the electrodes are deposited on top of the sensing layer. Due to the in-plane configuration of the electrodes, IDE sensors can accommodate sensing layers with a variety of morphologies, including rough layers with pinholes and particle coatings. The diffusion in the sensing layer is not obstructed by the top electrode, which can benefit the sensing of analytes with slow diffusion. However, IDE sensors have inferior sensitivity compared to MIM sensors due to the large stray contribution from the underlying substrate and surrounding atmosphere to the measured capacitance.

The heater provides the thermal perturbation to the sensing element, which results in out-of-equilibrium conditions required for detection of diffusion. The heater needs to provide a thermal input (either a pulse, step change, or a periodic temperature variation) that is fast relative to the time constant of diffusion of the fastest gas molecule to be detected. When the frequency of a periodic temperature variation is swept over a certain range, at least the fastest frequency in the range should fulfil this criterion. The thermal time constant of a whole sensor determines the lower detection limit for diffusivity and depends on the heater configuration, thermal properties, and thicknesses of layers composing the sensor.

In some embodiments, the heater is deposited on the backside of a thin substrate (FIG. 3a). In this configuration, the thickness of the substrate is significantly larger than the thickness of individual layers and dominates the thermal response of the sensor. The time constant is inversely proportional to the thermal conductivity of the substrate and proportional to its thickness. Therefore, electrical insulators with high thermal conductivity and suitable mechanical properties for thickness downsizing are desirable. The thermal time constant of an alumina substrate with 0.1 mm thickness is about 0.001 s⁻¹, and the thermal time constant of glass substrate of the same thickness is about 0.02 s⁻¹.

Ultra-low thermal time constants can be realized by using a suspended thin membrane (FIG. 3b). The membranes of sub-micrometre thickness can be readily fabricated using the microelectromechanical system (MEMS) fabrication process. The thermal time constant of a silicon nitride membrane with 1 μm thickness is about 10⁻⁷ s⁻¹.

The limit of detection at long times (low frequency) is determined by the maximum measurement time acceptable in sensing applications. Ideally, the measurement time should not exceed a few tens of seconds, for example, 10 seconds. The sensor bandwidth is determined by the range between the low and high time limits of the sensor. The typical bandwidth of diffusion discriminating sensors is between 10³ to 10⁷, with larger values preferred.

According to Equation 1, the intrinsic temporal response of the sensor depends on the diffusion constant of the VOC and the thickness of the nanoporous layer. The thickness of the nanoporous layer can be adjusted accordingly to the sensing application and may be, for example, less than 5 μm, suitably about 50 nm to 300 nm thick.

Implementation Variant with Separated Elements & Sampled Measurement

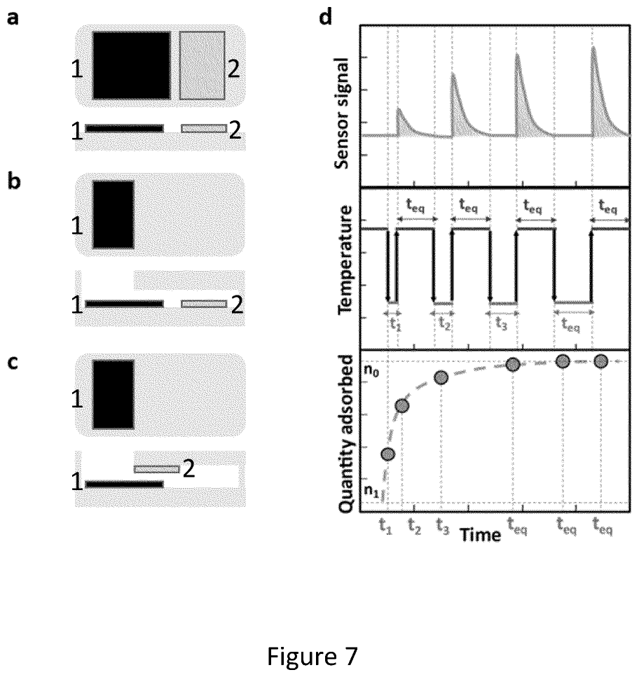

The heater with nanoporous material on top and the transduction element do not have to be stacked on top of each other. Instead, both elements can be spatially separated inside of an open-top or semi-enclosed sensor package (FIG. 7a-c). In this implementation, the measurement is performed in a sampled fashion. An example of this measurement method is given in FIG. 1d. In this example, the nanoporous material is first equilibrated with the gas stream at temperature T1. Next, the adsorbent temperature is rapidly changed to T0 (T0<T1). At this point, molecules will start to diffuse into the nanoporous material (since the adsorbed quantity n0>n1). Before equilibrium at T0 is established, the temperature jumps back to T1 after a well-defined dwell time t1, and the molecules that could diffuse in are desorbed. The desorbed fraction is quantified by the transduction element. By systematically varying the adsorption time t, the amount adsorbed for each dwell time will be determined and the amount adsorbed vs. time curve can be constructed and fitted with a suitable diffusion model to extract the diffusivity of the adsorbed molecule at T0 (FIG. 7d).

### Diffusion Measurement and Performance

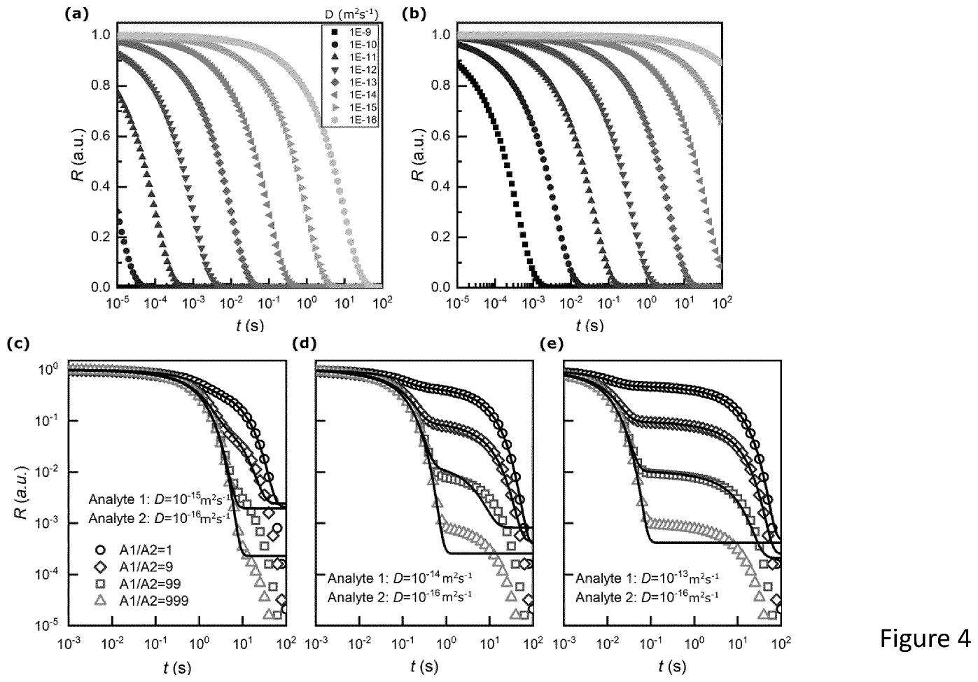

The diffusive transport of analyte molecules in the microporous material is largely controlled by steric interactions between the host and the analyte. Hence, diffusion kinetics are strongly dependent on the diameter of the diffusing molecule relative to the micropore size. Depending on the analyte molecule size, their diffusion constant can span over several orders of magnitude. FIG. 4 shows desorption profiles of analyte molecules with diffusion constants in the range from 10⁻⁹ m²s⁻¹ to 10⁻¹⁶ m²s⁻¹ for a 50 nm thick film (FIG. 4a) and a 1000 nm thick film of microporous material (FIG. 4b). Thinner films are preferred to detect analytes with smaller diffusion constants to reduce the time required for measurement. A sensor array composed of two or more sensing elements of different thicknesses can be used to extend the bandwidth of measurement.

The discrimination power of diffusion-based sensors is tested by examining the desorption profiles of binary mixtures of analytes with a factor of 10 (FIG. 4c), 100 (FIG. 4d), and 1000 (FIG. 4e) difference in diffusion constant. The different concentrations of both analytes are exemplified by comparing scenarios where the response due to the desorption of Analyte 2 is (i) equal, (ii) 9 times smaller, (iii) 99 times smaller, and (iv) 999 times smaller than the response due to the desorption of Analyte 1. In these examples, ‘A1/A2’ corresponds to the concentration ratio of both analytes. In the case of a factor of 10 difference in analyte diffusion constant, the double-exponential fitting is able to resolve the mixtures with A1/A2=1 ratio within 5% error and A1/A2=9 ratio within 50% error for Analyte 2. In the case of a factor of 100 difference in analyte diffusion constant, the sensor accuracy improves and could resolve A1/A2=1 and A1/A2=9 within 5% error and A1/A2=99 within 50% error for Analyte 2. In the case of a factor of 1000 difference in analyte diffusion constant, all A1/A2 ratios except A1/A2=9999 could be resolved within 5% error.

The proof of the concept is demonstrated using a MIM capacitance sensor that employs the prototypical microporous material, ZIF-8, as a sensing material. The diffusion constant of various analytes in ZIF-8 is given in Table 1. Typical interfering compounds in the ambient atmosphere, i.e., nitrogen, oxygen, carbon dioxide, and water, have diffusion constants smaller than 2×10⁻¹¹ m²s⁻¹. The analytes that can be considered as biomarkers in a human body, e.g., ethanol and acetone, have diffusion constants about 100 and 4000 times smaller than the background compounds, respectively. 1-hexane and trichloromethane, which are analytes of interest when monitoring the indoor air quality, have diffusion constants more than 100 000 times smaller than the background. These large differences in diffusion kinetics between background compounds and analytes of interest enable selective detection of analytes even at low concentrations of the latter.

In addition, several MOFs show a large difference in the equilibrium uptake of certain analytes, which can benefit the discrimination power of a sensor. The sensitivity of the sensor with the ZIF-8 layer in the MIM configuration toward different analytes is given in Table 2. ZIF-8 capacitive sensors show higher sensitivity toward 1-butanol and 1-hexane, which benefits the detection limit of these analytes. On the other hand, the sensor sensitivity toward water is small, which is an advantage since water is frequently an interfering compound.

TABLE 1
Diffusion constants of different analytes in ZIF-8 at 24° C.

| Compound | Diffusion constant (m²s⁻¹) |
|---|---|
| Oxygen (6) | 1 × 10⁻⁹ |
| Nitrogen (6) | 4 × 10⁻¹⁰ |
| Carbon dioxide (6) | 2 × 10⁻¹⁰ |
| Methane (6) | 4 × 10⁻¹¹ |
| Water (7) | 2 × 10⁻¹¹ |
| Methanol (8) | 4 × 10⁻¹¹ |
| Ethanol (7) | 2 × 10⁻¹³ |
| Acetone (10) | 5 × 10⁻¹⁵ |
| 1-propanol (10) | 1 × 10⁻¹⁵ |
| 1-butanol (10) | 3 × 10⁻¹⁶ |
| 1-hexane (10) | 2 × 10⁻¹⁶ |
| Trichloromethane (10) | 3 × 10⁻¹⁷ |

(6): [Hu et al. (2020) Inorg. Chem. Front. 7, 1598-1632]
(7): [Zhang et al. (2012) J. Phys. Chem. Let. 3 2130-2134]
(8): [Zhang et al. (2013) J. Phys. Chem. Let. 4, 3618-3622]
(10): [Measured using spectroscopic ellipsometry]

TABLE 2
Sensitivity (ΔC/C₀) of ZIF-8 sensors in MIM configuration toward
different analytes at 24° C. The values are measured in a
dilution regime before the characteristic sigmoidal uptake occurs.

| VOC | ΔC/C₀ per ppm (×10⁻⁶) |
|---|---|
| Water | 3.7 |
| MeOH | 3.9 |
| EtOH | 18 |
| Acetone | 91 |
| 1-PrOH | 100 |
| 1-BuOH | 630 |
| 1-hexane | 390 |

The data to support the above were collected using a capacitance sensor in the MIM configuration and a commercial hotplate setup that can reach heating rates of up to 150 K min-1. The sensor consists of a 260 nm thick ZIF-8 layer with a 20 nm thick silver top electrode. Capacitance measurements were performed at the frequency of 100 kHz. In pure nitrogen, the sensor shows the weak temperature dependence of capacitance with ΔC/C₀=2×10⁻⁴ per degree Celsius, which is due to the intrinsic temperature dependence of dielectric properties of ZIF-8. The change in capacitance upon applying a temperature step (as described in FIG. 1b) is instantaneous on a time scale achieved here and can be accounted for when the diffusion signal (S₂−S₁) is comparably small.

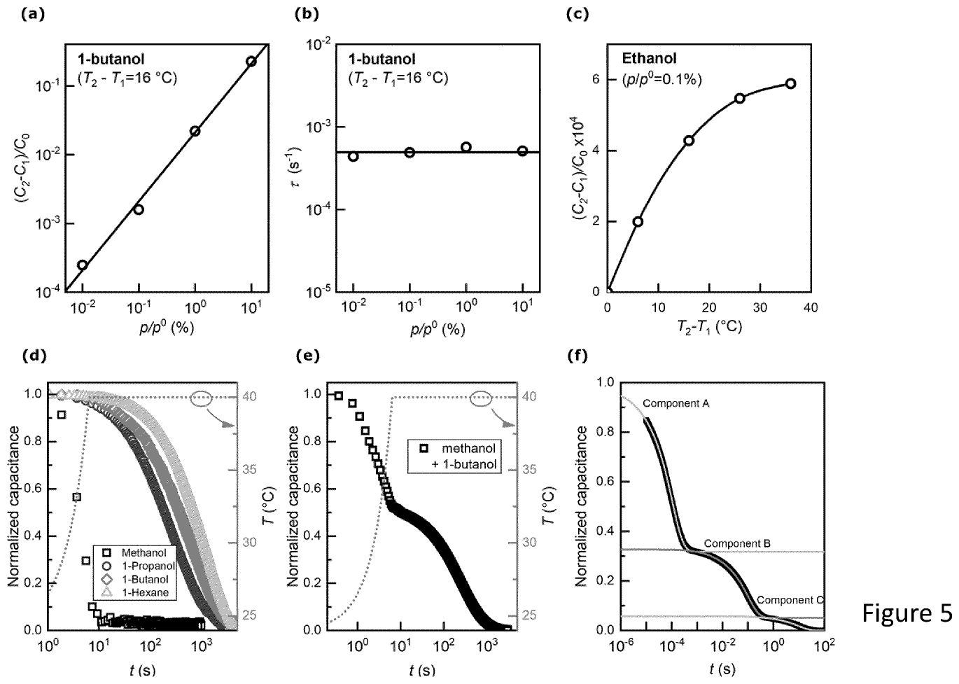

In the first example, the behaviour of diffusion signal at different concentrations of 1-butanol was tested (FIG. 5a). The magnitude of the diffusion signal scales linearly with the concentration of the analyte. Importantly, the ratios of sensitivity (ΔC/C₀) toward different analytes (Table 2) are equal when comparing equilibrium and diffusion signals, which indicates that the intrinsic selectivity of ZIF-8 is preserved in diffusion measurements. In the dilution regime, i.e., before the characteristic sigmoidal uptake occurs, the diffusion time constant is concentration-independent (FIG. 5b).

In the second example, the temperature step amplitude (T₂−T₁) was varied in the range between 6° C.-36° C. (FIG. 5c) and the diffusion signal (S₂−S₁) was monitored. The diffusion signal increases with the temperature step amplitude and shows an exponential decay dependence.

In the third example, the temperature step with the amplitude of 16° C. was applied in the presence of a single analyte (either methanol, water, ethanol, 1-propanol, 1-butanol, or hexane), and the sensor signal (capacitance) was monitored over time (FIG. 5d). The temporal response of a sensor to methanol, water, and ethanol is similar and follows the temperature perturbation, which indicates that these analytes diffuse too rapidly to be detected with a commercial hotplate setup. Other analytes show a step-wise response that is characteristic of diffusion-driven desorption. The fitting of the temporal sensor response yields the apparent diffusion constants of 6×10⁻¹⁷ m²s⁻¹, 3×10⁻¹⁷ m²s⁻¹, and 2×10⁻¹⁷ m²s⁻¹ for 1-propanol, 1-butanol, and 1-hexane, respectively. These apparent diffusion constants are about 10 times smaller than the values measured on thin films without the top electrode (measured optically by tracking refractive index upon thermal desorption of analyte). Importantly, the general trend of diffusion kinetics for different analytes is preserved.

In the fourth example, a temperature step with an amplitude of 16° C. was applied in the presence of a binary mixture of methanol (p/p⁰=8% at 24° C.) and 1-butanol (p/p⁰=0.5% at 24° C.). The temporal response of a sensor signal shows two desorption events occurring at the characteristic times (FIG. 5e). The first desorption event occurs at short times and follows the temperature perturbation, similar to pure methanol desorption in FIG. 5d. The second event occurs at much longer times and is comparable to the diffusion of pure 1-butanol in FIG. 5d.

In the fifth example, a desorption response of a sensor with an extended measurement bandwidth from 10⁻⁵ to 10² s is calculated for a ternary mixture of analytes. This scenario mimics a sensor fabricated on a suspended thin membrane with an ultra-low thermal time constant. The assumed mixture consisted of 40% of relative humidity, 200 ppm of acetone, and 10 ppm of 1-hexane. The sensor response is calculated using the sensor response values for single components given in Table 1 and Table 2. No mutual interactions between adsorbed analytes were assumed, which is reasonable assumption due to high dilution of analytes. Due to large differences in the diffusion constant of each analyte, three diffusion events are clearly observed in the calculated temporal response of a sensor. Each event can be fitted separately using a single exponential decay function to obtain the diffusion time constant and diffusion signal amplitude. Using this approach, the mixture composition can be perfectly reconstructed, i.e., the concentration and diffusion constant of individual analyte is measured within 5% error.

Another important feature of diffusion discriminating sensors is that the diffusion constants of analytes are intrinsic to a microporous material. This should simplify the transfer of calibration models between sensors, given that the microporous layer morphology and thickness are constant.

Additional measurements on single components and their mixtures were performed to evaluate the selectivity of the diffusion discriminating sensor in parts-per-million VOC concentration range. A classification of VOCs is possible by plotting the diffusion constant (giving the selectivity) vs the amplitude of the desorption curve (giving the concentration).

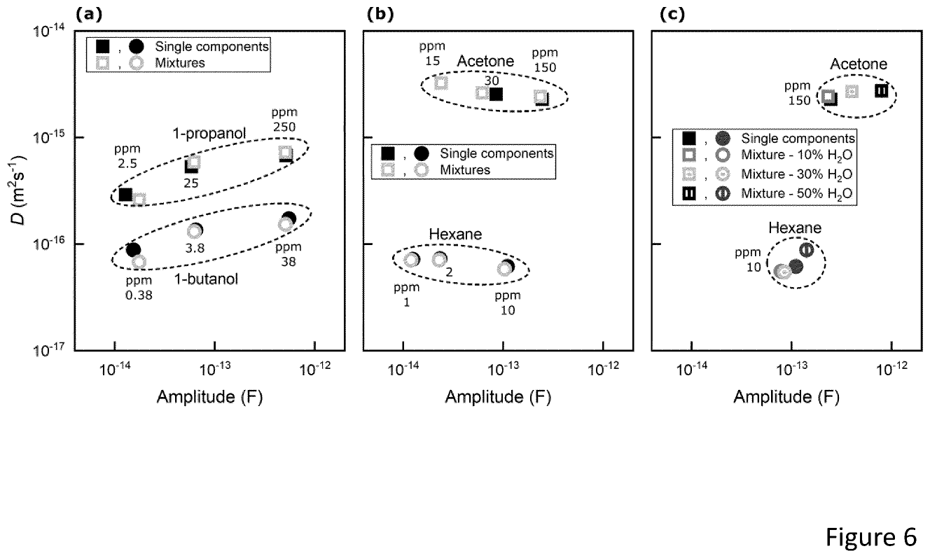

FIG. 6a shows the corresponding plots for pure 1-propanol and 1-butanol (full symbols) and binary mixtures of the two components (empty symbols). The ratio of 1-propanol and 1-butanol diffusion constants is in the range from 3 to 4. The two components can be clearly classified at all tested concentrations.

FIG. 6b shows the corresponding plots for pure acetone and hexane (full symbols) and binary mixtures of the two components (empty symbols). The ratio of 1-propanol and 1-butanol diffusion constants is in the range from 34 to 37. The two components can be clearly classified at all tested concentrations.

FIG. 6c shows the corresponding plots for pure acetone and hexane (full symbols) and ternary mixtures of the two components in presence of 10%, 30% or 50% of relative water humidity. The analysed ternary mixtures are similar to the theoretical mixture in FIG. 5f. Both acetone and hexane can be reliably detected in the interfering humid atmosphere.

1. A gas sensor for detecting one or a plurality of volatile compounds in a gas, the sensor comprising:
a signal transducer comprising a layer comprising nanopores wherein the gas has access to the nanopores in said layer,
a heating or cooling element, with adjustable temperature settings, for heating or cooling said layer comprising said nanopores,
means for access of said gas to said layer comprising said nanopores,
an electronic circuit, monitoring a time-dependent and a temperature-dependent signal generated by the signal transducer upon adsorption or release of a compound from the nanopores in said layer.

2. The gas sensor according to claim 1, wherein the layer comprising nanopores has a thickness of less than 5 μm, for example between 50 to 300 nm.

3. The gas sensor according to claim 1 or 2, wherein the nanopores in the layer comprising nanopores have an average diameter of below 10 nm, of below 10 nm, or of below 2 nm.

4. The gas sensor according to any one of claims 1 to 3, wherein the layer comprising nanopores is a zeolite or a porous carbon.

5. The gas sensor according to any one of claims 1 to 3, wherein the layer comprising nanopores is a MOF (Metal-Organic Framework).

6. The gas sensor according to any one of claims 1 to 5, wherein the heating element and layer comprising nanopores are separated by a heat conductive material.

7. The gas sensor according to claim 6, wherein the heat conductive material has a heat conductivity of at least 0.3 W/mK.

8. The gas sensor according to claim 6 or 7, wherein the heat conductive material is a non-electrically conductive material such as silicon nitride, silicon oxide, silicon carbide or a ceramic.

9. The gas sensor according to any one of claims 1 to 8, wherein the heating element is ohmic heater, such as a micro hotplate.

10. The gas sensor according to any one of claims 1 to 9, wherein the signal transducer is an electronic, capacitive, optical or gravimetric signal transducer.

11. The gas sensor according to claim 10, wherein the capacitive signal transducer comprises a bottom heat conductive layer, and a top gas permeable conductive layer, and the layer comprising nanopores is positioned between said bottom layer and said top layer.

12. The gas sensor according to claim 10, wherein the optical signal transducer a bottom reflective or semi-reflective layer, a top reflective or semi-reflective gas permeable layer and the layer comprising nanopores is positioned between said bottom and said top layer.

13. The gas sensor according to claim 10, comprising a gravimetric signal transducer wherein the layer comprising nanopores is positioned, and in contact with, on one or more mechanical resonators of which the resonant frequency or amplitude can be monitored.

14. The gas sensor according to claim 13, wherein the gravimetric transducer operates in a static or resonant mode.

15. The gas sensor according to claim 13 or 14, wherein the gravimetric transducer is a cantilever or a coupled resonator.

16. The gas sensor according to any one of claim 1 or 15, wherein the layer comprising the nanopores is in direct contact with the transducer.

17. The gas sensor according to any one of claim 1 or 15, wherein the layer comprising the nanopores and the transducer are spatially separated.

18. The gas sensor according to claim 17, wherein the transducer is a metal oxide semiconductor sensor.

19. The sensor according to any one of claims 1 to 18, wherein the gas sensor comprises a plurality of layers comprising nanopores, wherein the material of the layers have different affinities for a volatile compound and/or different diffusion properties for a volatile compounds, and wherein each of layers is part of an individual signal transducer.

20. The sensor according to claim 19, wherein each of the plurality of layers comprising nanopores can be subjected to a separate temperature regime.

21. The sensor according to claim 19 or 20, wherein each of the plurality of layers comprising nanopores differs in thickness.

22. A method for determining the presence and/or quantity of a plurality of volatile compounds in a gas comprising the steps of:
a) Introducing a gas into a sensor in accordance to any one of claims 1 to 20 whereby compounds in the gas can adsorb in the nanopores of the layer comprising nanopores,
b) decreasing or increasing the temperature of the layer comprising nanopores, thereby releasing adsorbed compounds from the nanopores upon heating, or adsorbing compounds in the nanopores upon cooling,
c) measuring from the signal transducer the temperature-dependent and time dependent release and/or adsorption of compounds from or to the nanopores,
d) determining based on the measurements of the transducer the presence and/or concentration of at least two volatile compounds in the gas.

23. The method according to claim 22, wherein is step b) the temperature of the layer comprising nanopores is increased, thereby releasing adsorbed compounds from the nanopores.

24. The method according to claim 22 or 23, determining in step d) the presence and concentration of water in the gas.

25. The method according to any one of claims 22 to 24, wherein the temperature of the layer comprising nanopores is perturbated in a periodic manner.

26. The method according to any one of claims 22 to 25, wherein the adsorption or release of compounds is monitored on multiple layers comprising nanopores.

27. The method according to any one of claims 22 to 26, wherein the gas introduced in step a) contains up to 50% (v/v) up to 75% (v/v) or up to 100% (v/v) water vapor.

28. The method according to any one of claims 22 to 27, wherein the gas introduced in step a) is outside ambient air or air within a building.

29. The method according to any one of claims 22 to 28, wherein the gas introduced in step a) is an exhaled animal or human breath.

30. The method according to any one of claims 22 to 29, wherein the method determines the presence and/or concentration of one or more 1-propanol, 1-butanol, acetone pentane and hexane in a gas.

31. The method according to any one of claims 22 to 30, wherein the method determines the presence and/or concentration of one or more 1-propanol, 1-butanol, acetone pentane and hexane in a gas comprising water vapor.
